(19) United States
(12) Patent Application Publication
(10) Pub. No.: US 2015/0250765 A1
(43) Pub. Date: Sep. 10, 2015

(54) NITAZOXANIDE AND MEBENDAZOLE SYNERGIC COMPOSITION, PROCESSES FOR THE PREPARATION THEREOF, AND USE OF SAID COMPOSITION FOR THE TREATMENT OF HUMAN PARASITOSIS

(71) Applicant: SIEGFRIED RHEIN S.A. DE C.V., Delg. Alvaro Obregon (MX)

(72) Inventor: Esteban Alejandro Fiore, Ciudad de Buenos Aires (AR)

(21) Appl. No.: 14/431,623

(22) PCT Filed: Sep. 27, 2012
(86) PCT No.: PCT/IB2012/055166

§ 371 Date: Mar. 26, 2015

(51) Int. Cl. A61K 31/4184; A61K 9/20; A61K 31/426

(52) U.S. Cl. CPC A61K 31/4184; A61K 31/426; A61K 9/2095

(57) ABSTRACT

The invention relates to a synergic pharmaceutical composition of mebendazole (MBDZ) with nitazoxanide (NTZX) for the treatment of human parasitosis, which significantly increases the spectrum of MBDZ against protozoa and reinforces the anthelmintic action of NTZX. Furthermore, when said pharmaceutical combination of MBDZ and NTZX is used, the same effects of the individual active substances are maintained in the systemic action of the active metabolite of NTZX, tizoxanide, and in the treatment of some systemic forms of parasitosis. In addition, the synergic pharmaceutical combination of MBDZ with NTZX achieves a larger anti-parasite spectrum, while maintaining the efficiency and safety profiles of both active substances independently. All of these effects enable the differentiation of the pharmaceutical combination from the rest of the conventional anti-parasite treatments that have more limited spectra. The invention therefore relates principally to the synergic effects of the pharmaceutical combination of MBDZ and NTZX.

### TECHNICAL FIELD OF THE INVENTION

This invention relates to a synergic combination of nitazoxanide and mebendazole for the treatment of human parasitosis caused by protozoa and helminths.

This association combines the nitazoxanide and mebendazole compounds, showing an unexpected synergism, widening the spectrum and enhancing the antihelmintic and antiprotozoal action of both active ingredients.

### BACKGROUNDS OF THE INVENTION

Intestinal parasitic infections are among the most significant causes of morbidity and mortality, particularly in developing countries. Helminth infections are a public health problem worldwide. For example, helminthiasis affect chronically about one third of the world population, with an estimated one million cases of geothelminths, 900 million prevalent cases of trichuriasis, and 500 million cases of anclyostoma.

Parasitic infections affect mainly children of school age and are often transmitted where hygiene/sanitation are poor. This child population affected by intestinal parasites is due to their immunological immaturity and the poor development of hygiene. These parasitosis can lead to negative consequences, both physical as from the cognitive point of view, in many parasitized children.

Most parasites are transmitted by fecal-oral route, particularly by ingestion of water and/or food contaminated with the various infectious forms of the parasites. Geohelminths require a maturation process in the soil to infect another host and can do so through larvae that penetrates the skin.

A feature of the incidence of parasites in school children is the high incidence of infection of more than one species. In an epidemiological study conducted in the province of Mendoza, Argentina, an overall prevalence of intestinal parasites of 80.5% was observed, with values ranging from 88% (age group 5-10 years) and 63.8% (age group of 11-14 years), where 37.6% of positive presented a single species, while in the rest parasitic associations of up to 4 different genera were found [Salomón, M. C. et al. Parasitol. Latinoam. V.62 n.1-2 Santiago, June 2007].

An ideal parasite is one that proves to have a wide range to cover as many intestinal parasites (helminths and protozoa) as possible, easy delivery scheme; good biosafety profile in both children and adults and also that in the cost-benefit analysis justifies its use in the population scope.

There have been various studies regarding the associations of different antiparasitics, as in the case of a clinical trial conducted with the association of albendazole with praziquantel against trichuris-trichiura, where no synergic effect was observed for both drugs [Sirivichayakul, C. et al., Southeast Asian J. Trop. Med. Public Health. 2001 32: 297-301]. Neither synergism was observed in the treatment of geothelmiths and schistosomiasis in school children with the albendazole and praziquantel combination [Olds, G. R. et al, 1999, J. Infect. Diseases 179: 996-1003), as is described in a study where albendazole (500 mg) and mebendazole (400 mg) were administrable as a single dose for the treatment of ancylostoma and other helminths and in 200 infected children [Soukhathammavong, P A. Et. al, PLoS Negl Trop Dis. 2012 January; 6(1):e1417. Epub 2012 Jan 3], where no synergic effect was observed in the cure rate against ancylostoma. In another epidemiological study performed in northeastern Argentina, it was found that 74% of the children were poly parasitized. The most frequent combination found was Enterobius vermicularis, Blastocystis hominis and/or Giardia [Milano, A. et al., Enteroparasitosis infantil intestinal en Argentina. Medicina (Buenos Aires) 2000; 60: 23-4].

The compound nitazoxanide was disclosed as a product in the U.S. Pat. No. 3,950,351 and its equivalents, whose owner is S.P.R.L. Phavic and the priority date is Aug. 8, 1973. Then, the U.S. Pat. No. 5,387,598 owned by Romark and with priority date Apr. 13, 1994 describes a formulation containing Nitazoxanide and Tizoxanide.

The Mebendazole compound was described as a product in the U.S. Pat. No. 3,657,267 and their equivalents, owned by Janssen Pharmaceutica N.V. and which priority date is Jun. 20, 1969.

Due to the resistance growing development to antiparasitics by the intestinal nematodes, it was found that it was necessary to investigate new alternative strategies to the pharmacological control of parasitosis diseases. Combining different antiparasitics was considered a strategy to reach a wider action spectrum and enhance the anthelmintic and antiprotozoal action of both active ingredients.

### BRIEF DESCRIPTION OF THE FIGURE

FIG. 1 illustrates the evaluation results of L4 helminth larvae.

### DETAILED DESCRIPTION OF THE INVENTION

By associating Mebendazole (MBDZ) with Nitazoxanide (NTZX), it was surprisingly achieved to significantly broaden the spectrum of MBDZ against protozoa and strengthen NTZX anthelmintic action. In addition, the systemic action of the active metabolite of the NTZX, tizoxanide, and the treatment of some systemic forms of parasitosis are maintained by using the pharmaceutical combination of MBDZ and NTZX. Further, the synergic effects of the pharmaceutical combination of MBDZ and NTZX achieve a wider antiparasitic spectrum, while maintaining effectiveness and safety profiles of both active ingredients separately. All these effects allow this invention to differ from other typical antiparasitic treatments with narrower spectra. The synergic effects of the pharmaceutical combination of MBDZ and NTZX are, therefore, the main objective of this invention.

Therefore, this invention relates to a synergic pharmaceutical combination for the treatment of human parasitosis comprising a therapeutically effective amount of Nitazoxanide antiparasitic and a therapeutically effective amount of Mebendazole antiparasitic.

In another aspect, the invention relates to a pharmaceutical composition for oral delivery for the treatment of human parasitosis comprising the combination of a therapeutically effective amount of Nitazoxanide antiparasitic with a therapeutically effective amount of Mebendazole antiparasitic, along with pharmaceutically acceptable excipients.

In another aspect, the invention relates to a pharmaceutical composition for oral delivery for the treatment of human parasitosis comprising the combination of a therapeutically effective amount of Nitazoxanide antiparasitic with a therapeutically effective amount of Mebendazole antiparasitic, along with pharmaceutically acceptable excipients, wherein such pharmaceutical composition for oral delivery may be a coated tablet.

In a further aspect, the invention relates to a pharmaceutical composition for oral delivery for the treatment of human parasitosis comprising the combination a therapeutically effective amount of Nitazoxanide antiparasitic with a therapeutically effective amount of Mebendazole antiparasitic, with pharmaceutically acceptable excipients, wherein such pharmaceutical composition for oral delivery may be a powder for extemporaneous reconstitution.

Furthermore, in another aspect, the invention relates to a pharmaceutical composition for oral delivery for the treatment of human parasitosis comprising the combination of a therapeutically effective amount of Nitazoxanide antiparasitic with a therapeutically effective amount of Mebendazole antiparasitic, along with pharmaceutically acceptable excipients, wherein such pharmaceutical composition for oral delivery may be administrable once or twice a day.

The therapeutically effective doses of Nitazoxanide used in the oral delivery pharmaceutical composition of the invention may be comprised within the range of 50 mg to 1200 mg and the therapeutically effective doses of mebendazole in the range of 20 to 500 mg, preferably containing, per adult dosage unit, 500 mg of nitazoxanide and 100 mg of Mebendazole. The powder for extemporaneous reconstitution used for the pediatric formulation preferably contains 100 mg of nitazoxanide and 50 mg Mebendazole. Finally, the oral delivery pharmaceutical composition of the invention which is administered once a day preferably comprises 1000 mg of Nitazoxanide and 200 mg of Mebendazole.

In a further aspect, this invention further relates to processes for preparing the pharmaceutical composition for oral delivery which comprises granulating, mixing and tableting therapeutically effective amounts of the active ingredients nitazoxanide and mebendazole, along with pharmaceutically acceptable excipients and optionally coating the tablets obtained.

In another aspect, this invention relates to processes for preparing the pharmaceutical composition of pediatric oral delivery which comprises the mixture of therapeutically effective amounts of the active ingredients with Nitazoxanide and Mebendazole along with pharmaceutically acceptable excipients. The powder for extemporaneous reconstitution is thus obtained.

A further object of this invention is the use of a therapeutically effective amount of the Nitazoxanide antiparasitic along with a therapeutically effective amount of the Mebendazole antiparasitic in the manufacture of a pharmaceutical composition for oral delivery for the treatment of human parasitosis, particularly for the treatment of human parasitosis caused by protozoa and helminths.

The synergism has been demonstrated by in vitro studies showing the anthelmintic and antiprotozoal action of the pharmaceutical combination of the present invention according to that described in example 4:

### WORKING EXAMPLES

### Example 1

The process for preparing a formulation of coated tablets for delivery every 12 hours is described.

For coated tablet

| | |
|---|---|
| mg | % |

CORE

| | | |
|---|---|---|
| Nitazoxanide | 500.00 | 53.76 |
| Mebendazole | 100.00 | 10.75 |
| Corn starch | 40.00 | 4.30 |
| Pregelatinized starch | 70.00 | 7.53 |
| Povidone K30 | 40.00 | 4.30 |
| Microcrystalline cellulose | 91.00 | 9.79 |
| Sodium glycolate starch | 43.00 | 4.62 |
| Talc | 8.00 | 0.86 |
| Magnesium stearate | 8.00 | 0.86 |
| Purified water | Approx. 182 | |

COATING

| | | |
|---|---|---|
| Hydroxypropylmethylcellulose | 17.00 | 1.83 |
| Titanium dioxide | 9.00 | 0.97 |
| Triacetine | 3.50 | 0.38 |
| Yellow iron oxide | 0.50 | 0.05 |
| Purified water | Approx. 220 | |

### Method of Preparation

1. Solve Povidone K30 in purified water.
2. Sieve through a 1 mm mesh and transfer to the mixer: nitazoxanide, nebendazole, corn starch and pregelatinized starch.
3. Mix for 2 minutes and add the solution obtained in item 1.

Amass until the point of granulation is obtained.
4. Calibrate the granulate through a 3 mm mesh.
5. Dry the calibrated granulation until reaching a 2 to 4% moisture.
6. Calibrate the granulate through a 1 mm mesh.
7. Mix the calibrated granulate with the microcrystalline cellulose, the sodium glycolate starch, the talc and the magnesium stearate.
8. Tablet to 900 mg±5% mg of theoretical weight.
9. Prepare the coating by perfectly suspending the yellow iron oxide and the titanium dioxide in the water and subsequently adding hydroxypropylmethylcellulose and triacetine.
10. Coat the tablets until a theoretical weight of about 930 mg±5% is achieved.

### Example 2

The details of the preparation process of the powder for oral suspension for pediatric use deliverable every 12 hours are shown below.

34 g of powder for obtaining
100 ml of reconstituted
suspension contain

| | g | % |
|---|---|---|
| Nitazoxanide | 2.000 | 5.882 |
| Mebendazole | 1.000 | 2.941 |
| Sodium benzoate | 0.200 | 0.588 |
| Refined sugar | 29.599 | 87.056 |
| Xanthan Gum | 0.800 | 2.353 |
| Anhydrous citric | 0.200 | 0.588 |
| acid | | |
| Dihydrate sodium | 0.050 | 0.147 |
| citrate | | |
| FD & C Red 40 dye | 0.001 | 0.003 |
| Essence of | 0.150 | 0.441 |
| strawberry powder | | |
| | 34.000 | 100.00 |

### Method of Preparation

1. Grind the sugar to fine powder, set aside 10% of the grinding, put the rest in a suitable mixer.
2. Mix the FD & C red 40 dye, the powdered strawberry Essence and the xanthan gum, grind the mixture to a fine powder. Add it to the mixer of item 1. Mix for 5 minutes.
3. Add the previously milled to a fine powder nitazoxanide, mebendazole, sodium benzoate, dihydrate sodium citrate, anhydrous citric acid to the mix of item 1. Use the set aside 10% of sugar to rinse the equipment where the grindings were performed and add it to the mixer. Mix for 15 minutes.
4. Pack 34 g per flask.
5. Prior to use, reconstitute with 100 ml of drinking water.

### Example 3

The process for preparing a formulation of coated tablets deliverable every 24 hours is described.

Per coated tablet

| | |
|---|---|
| mg | % |

CORE

| | | |
|---|---|---|
| Nitazoxanide | 1000.00 | 56.82 |
| Mebendazole | 200.00 | 11.36 |
| Corn starch | 70.00 | 3.98 |
| Pregelatinized starch | 130.00 | 7.39 |
| Povidone k30 | 75.00 | 4.26 |
| Microcrystalline cellulose | 120.00 | 6.82 |
| Sodium glycolate starch | 75.00 | 4.26 |
| Talc | 15.00 | 0.85 |
| Magnesium stearate | 15.00 | 0.85 |
| Purified water | Aprox. 363 | |

COATING

| | | |
|---|---|---|
| Hydroxypropylmethylcellulose | 34.00 | 1.93 |
| Titanium dioxide | 18.00 | 1.02 |
| Triacetine | 7.00 | 0.40 |
| Yellow iron oxide | 1.00 | 0.06 |
| Purified water | Aprox. 440 | |

### Method of Preparation

1. Solve povidone K30 in purified water.
2. Sieve through a 1 mm mesh and transfer to the mixer:
3. Nitazoxanide, mebendazole, corn starch and pregelatinized starch
4. Mix for 2 minutes and add the solution obtained in item 1. Amass until the granulation point obtention.
5. Calibrate the granulate through a 3 mm mesh.
6. Dry the calibrated granulate until a 2 to 4% moisture is obtained.
7. Calibrate the dry granulate through a 1 mm mesh.
8. Mix the calibrated granulate with the microcrystalline cellulose, the sodium glycolate starch, the talc and the magnesium stearate.
9. Tablet to 1.700 mg±5% mg of theoretical weight.
10. Prepare the coating by perfectly suspending the yellow iron oxide and titanium dioxide in the water and subsequently adding hydroxypropylmethylcellulose and triacetine.
11. Coat the tablets until obtaining a theoretical weight of 1760 mg±5%.

### Example 4

### In Vitro Tests

In vitro tests were performed to assess and know the susceptibility and resistance of various parasites to the pharmaceutical combination of MBDZ and NTZX.

### Methods:

### A) Anthelmintic In Vitro Determination:

1) In order to determine the in vitro anthelmintic effectiveness of the pharmaceutical combination of NTZX and MBDZ the “inhibition of larval migration test” was used, which was developed by Wagland et al. (1992) and modified by Rable et al. (1994) and Paolini et al. 2004). The objective of this study was to use the inhibition of larval migration test to study the anthelmintic effect of the two active ingredients separately and in combination, in the migration of L4 (larva or adult worm) of different kinds of helminths (eg. trichuris spp), in doses of 100 μg/ml (100 μg/ml NTZX, 100 μg/ml MBDZ and 100 μg/ml of the combination [50 μg/ml NTZX and 50 μg/ml MBDZ]).

The rats of the Sprague-Dawley vivarium strain, parasites naive, were infected with different kinds of helminths (eg. Trichuria trichuris infected eggs). Approximately 3 g of feces were placed in culture bottles. The feces were cultured to produce worms or larvae (L4 stage) at a temperature of 37° C. for 72 hours. In order to determine the in vitro anthelmintic effectiveness the larval migration inhibition test was used. For such test, 15 mm long by 10 mm diameter filters with transparent acrylic tubes were built, to which a nylon mesh of 20 microns was adhered on one end. 2 ml plastic tubes were used, to which the subject active ingredients (100 μg/ml NTZX, 100 μg/ml MBDZ and 100 mg/ml of the combination of NTZX and MBDZ and a control with no active drugs) were added, diluted in 0.4 ml and 100 to 200 L4 and suspended in 0.1 ml of aqueous solution. The tubes were incubated at 37° C. for 16 hours. The total content of these tubes, 0.5 ml, was transferred to the filters located within the acrylic tubes and grown at room temperature for 18 hours to allow the larvae to migrate through the filters within the chamber. Then, the filters were removed from the chambers and the total number of larvae in the filters and in chambers was counted.

The larval migration inhibition (LMI) was determined using the following formula:

$$L M I = \frac{A - B}{A} \times 100$$

Wherein A=larvae migrated ratio in the control and B=larvae migrated ratio in the treatments.

The larval migration inhibition test results obtained were as follows:
Control Group: there was a 100% parasites migration, therefore the LMI=0.
Group treated with NTZX: there was a 40% migration, therefore the LMI=60%.
Group treated with MBDZ: there was a 30% migration, therefor the LMI=70%.
Group treated with the combination (NTZX+MBDZ): there was a 10% migration, therefore the LMI=90%.

2) Another method for the larval motility assessment was using the “scale of larval motility” in the various tubes with the different drugs and the control group. The scale of motility is a scale from 0 to 3 points, wherein 0=death; 1=very low motility; 2=low motility and 3=normal motility.

The following are the results obtained by this method:

1) Control culture, with no active drug: complete parasite viability at 72 hours. Motility scale=3
2) Culture with NTZX: Motility Scale=2 in all observed parasites.
3) Culture with MBDZ: Motility Scale=1 in all observed parasites.
4) Culture with the NTZX+MBDZ combination=0 in all observed parasites.

Based on the above assessed inhibition test, it is shown that the combination of the drugs NTZX and MBDZ has a greater synergic effect on helminthes L4 than that observed with the two active ingredients NTZX and MBDZ separately.

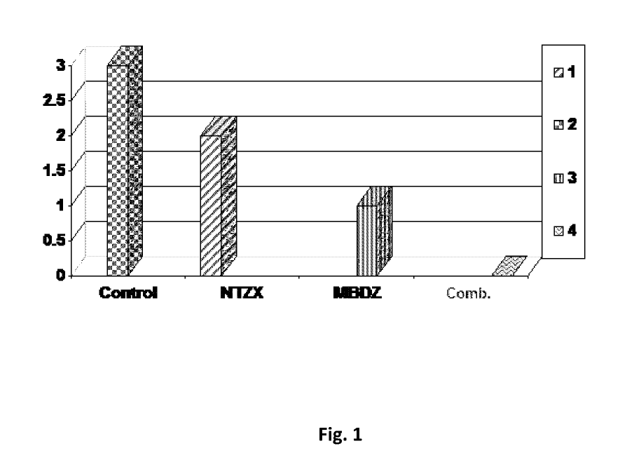

FIG. 1 illustrates the results of the L4 helminth larvae assessment, wherein: control=3; NTZX=2; MBDZ=1; Combination (NTZX+MBDZ)=0. Motility scale: 0=death; 1=very low motility; 2=low motility; 3=normal motility.

### B) Antiprotozoal In Vitro Determination:

1) The in vitro antiprotozoal effectiveness of the pharmaceutical combination of NTZX with MBCZ was determined in an intestinal Giardia culture as compared with the NTZX and MBDZ active ingredients separately. In this study the morphology, the adhesion and the viability of the Giardias trophozoites was assessed in in vitro cultures.

### Materials and Method:

The antiparasitic agents used were NTZX at a 2 μg/ml concentration; MBDZ at a 2 μg/ml concentration and a pharmaceutical combination of 1 μg/ml NTZX and 1 μg/ml MBDZ. The trophozoites were obtained from the intestines of rats (Sprague-Dawley vivarium strain) previously infected with Giardia intestinalis.

The trophozoites were isolated in a BI-S33 culture media containing 10% bovine serum without added antibiotic. From this media, 4.5 ml were extracted, which were separated in 8 glass tubes with screw caps. These tubes (labeled with the active ingredients separately, the combination and other as control) were inoculated with the corresponding active ingredients, leaving the control drug naive. 4 of the tubes were exposed to the drugs for 4 hours and the other 4 tubes were exposed to the drugs for 24 hours. After the established periods of time were over (4 to 24 hours) the tubes underwent centrifugation (5 minutes at 500 rpm), where the supernatant was extracted and removed. The decanted material was then dyed with a 0.1% eosin solution to assess the viability of trophozoites:
a. The adhesion and growth uniformity of trophozoites was assessed, using an optical microscope in 5-10 fields (×100 and ×200). For this, a table was drafted describing the following:

| | Number of adhered |
|---|---|
| Ranking | trophozoites |
| 0 | None |
| 1 | <1 |
| 2 | 1-10 |
| 3 | 10-25 |
| 4 | 25-50 |
| 5 | 50-100 |
| 6 | 100-250 |
| 7 | 250-500 |
| 8 | 500-1000 |
| 9 | 1000-2000 |
| 10 | clusters |

b. The trophozoites viability rate was assessed, by using a Neubauer camera to the optical microscope. The percentage of non-viable trophozoites was estimated as compared with the total state of trophozoites (live and immobile).
c. The mobility and morphology of the trophozoites was assessed. Mobility is divided into:

| Ranking | Mobility |
|---|---|
| 3 | Progressive, fast or |
| | slow |
| 2 | Non progressive or in |
| | situ |
| 1 | immobile |

The normal morphology of trophozoites under the optical microscope is characterized by the following: unicellular organism, with pyriform morphology, bilateral symmetry, flagellated, bi-nucleated (vacuolar complex) on its dorsal face. Any change in morphology was correspondingly detailed.

Results:

Based on the microscopic morphological changes following exposure to the different active ingredients, the following was found:
It was observed that the morphological changes were correlated with loss of viability thereof in the group treated with the combination of both active ingredients after 24 hours of exposure. The main changes were: total loss of their pyriform shape, where many folded trophozoites were observed; loss of the bilateral symmetry and of the dorsal vacuolar complex; almost total immobility of the trophozoites. Adhered trophozoites were observed in a covered or almost completely covered field, forming nests or flaps.
In cases where they were exposed to NTZX for 24 hours, globular trophozoites (very enlarged), a moderate amount of folded trophozoites and partial loss of dorsal vacuolar complex were observed. A low number of motile trophozoites (mostly in situ) and erratic movements were observed. The amount of adhered trophozoites was moderate.
In cases of MBDZ exposure for 24 hours, it was observed that some trophozoites were globular, some folded, some of distorted forms (partial loss of bilateral symmetry).
In the control group, no changes were observed in the pyriform shape nor in the progressive motility of the trophozoites, same maintaining the flagella and the dorsal vacuolar complex.

| | | | Rank- | | | |
|---|---|---|---|---|---|---|
| Hours | | Non | ing of | Via- | Mo- | |
| of drug | | viable | tropho- | bility | bility | Morpho- |
| expo- | | Tropho- | zoites | rate | Rank- | logical |
| sure | Drugs | zoites % | adhesion | (%) | ing | changes |
| 4 | Control | 0 | 0 | 100 | 3 | No |
| hours | | | | | | changes |
| | DZ | 30 | 4 | 70 | 2 | |
| | NTZX | 43.5 | 5 | 56.5 | 2 | |
| | MBDZ + | 66.5 | 7 | 33.5 | 1 | |
| | NTZX | | | | | |
| 24 | Control | 0 | 0 | 100 | 3 | No |
| hours | | | | | | changes |
| | MBDZ | 48 | 5 | 52 | 1 | |
| | NTZX | 67.5 | 7 | 32.5 | 1 | |
| | MBDZ + | 93.7 | 10 | 6.3 | 1 | |
| | NTZX | | | | | |

The above in vitro tests show the antiparasitic superiority, in helminth and protozoan, in both, the active ingredients separately (MBDZ and NTZX) and in the pharmaceutical combination of MBDZ with NTZX. Also, these tests show a synergic effect of the pharmaceutical combination of MBDZ with NTZX on the two active ingredients MBDZ and NTZX separately. The antigiardial effect of MBDZ is enhanced by the combination with NTZX, and the same happens with the antiprotozoal effect of NTZX when it is combined with MBDZ.

1. A synergic pharmaceutical composition adapted to be orally administrable as antiparasitic comprising the combination of a therapeutically effective amount of the nitazoxanide antiparasitic with a therapeutically effective amount of mebendazole antiparasitic, along with pharmaceutically acceptable excipients.

2. A pharmaceutical composition adapted to be orally administrable according to claim 1, wherein the pharmaceutical composition is a coated tablet.

3. A pharmaceutical composition adapted to be orally administrable according to claim 1, wherein the pharmaceutical composition is a powder for extemporaneous reconstitution.

4. A pharmaceutical composition adapted to be orally administrable according claim 1, wherein the therapeutically effective amount of nitazoxanide ranges between 50 mg and 1200 mg and the therapeutically effective amount of mebendazole ranges between 20 mg and 500 mg.

5. A pharmaceutical composition adapted to be orally administrable according to claim 4, wherein the therapeutically effective amount of nitazoxanide is 500 mg per dosage unit and the therapeutically effective amount of mebendazole is 100 mg per dosage unit.

6. A pharmaceutical composition adapted to be orally administrable according to claim 4, wherein said pharmaceutical composition is for pediatric use and wherein the therapeutically effective amount of nitazoxanide is 100 mg per dosage unit and the therapeutically effective amount of mebendazole is 50 mg per dosage unit.

7. A pharmaceutical composition adapted to be orally administrable according to claim 4, wherein the pharmaceutical composition is further adapted to be administrable every 12 hours.

8. A pharmaceutical composition adapted to be orally administrable according to claim 4, wherein the pharmaceutical composition is further adapted to be administrable once a day.

9. A pharmaceutical composition adapted to be orally administrable according to claim 8, wherein the therapeutically effective amount of nitazoxanide is 1000 mg and the therapeutically effective amount of mebendazole is 200 mg per dosage unit.

10. A pharmaceutical combination for the treatment of human parasitosis according to claim 1, comprising a therapeutically effective amount of nitazoxanide antiparasitic and a therapeutically effective amount of mebendazole antiparasitic.

11. A process for preparing a pharmaceutical composition adapted to be orally administrable according to claim 1, comprising granulating, mixing and tableting a therapeutically effective amount of nitazoxanide antiparasitic with a therapeutically effective amount of mebendazole antiparasitic along with pharmaceutically acceptable excipients.

12. A process for preparing the pharmaceutical composition adapted to be orally administrable according to claim 11, further comprising coating the obtained pharmaceutical composition.

13. A process for preparing a pharmaceutical composition of extemporaneous reconstitution adapted to be orally administrable according to claim 3, comprising mixing a therapeutically effective amount of nitazoxanide antiparasitic with a therapeutically effective amount of mebendazole antiparasitic, along with pharmaceutically acceptable excipients.

14. The use of a therapeutically effective amount of nitazoxanide antiparasitic in combination with a therapeutically effective amount of mebendazole antiparasitic in the manufacture of a medicament for the treatment of human parasitosis.

15. The use according to claim 14, wherein the medicament is for treating human parasitosis caused by protozoa and helminths.
